(19) United States
(12) Patent Application Publication
(10) Pub. No.: US 2022/0313364 A1
(43) Pub. Date: Oct. 6, 2022

(54) DEVICES AND METHODS FOR TARGETING IMPLANT DEPLOYMENT IN TISSUE

(71) Applicant: Teleflex Life Sciences Limited, Valletta (MT)

(72) Inventors: Brian R. DuBois, Redwood City, CA (US); Barel Levy, Jerusalem (IL); Lior Zfira, Jerusalem (IL); Daniel Tomer, Jerusalem (IL); Moshe Safran, Jerusalem (IL)

(73) Assignee: Teleflex Life Sciences Limited, Valletta (MT)

(21) Appl. No.: 17/615,934

(22) PCT Filed: Jun. 18, 2020
(86) PCT No.: PCT/US2020/038554

§ 371 Date: Dec. 2, 2021

Related U.S. Application Data
(60) Provisional application No. 62/863,305, filed on Jun. 19, 2019.

(51) Int. Cl. A61B 34/20; A61B 17/34; A61B 90/00

(52) U.S. Cl. CPC A61B 34/20; A61B 17/3403; A61B 90/37; A61B 2090/363; A61B 2017/00128

(57) ABSTRACT

A system and method for providing relative positional information of a therapeutic or diagnostic device during the treatment of urinary tract diseases and conditions.

### BACKGROUND OF THE INVENTION

The present invention relates generally to medical devices and methods of use, and more particularly to systems and methods for conveying positional information and thereby improving the selection of target sites for deploying anchors and/or implants into tissue for the treatment of urinary tract diseases and conditions.

Benign Prostatic Hyperplasia (BPH), or prostate gland enlargement, is one of the most common medical conditions that affect men, particularly elderly men. It has been reported that, in the United States, more than half of all men have histopathologic evidence of BPH by age 60 and, by age 85, approximately 9 out of 10 men suffer from the condition. Moreover, the incidence and prevalence of BPH are expected to increase as the average age of the population in developed countries increases.

The prostate gland enlarges throughout a man's life. In some men, the prostatic capsule surrounding the prostate gland prevents the prostate gland from enlarging further. This causes the inner region of the prostate gland to squeeze the urethra. This pressure on the urethra increases resistance to urine flow through the region of the urethra enclosed by the prostate. Therefore, the urinary bladder has to exert more pressure to force urine through the increased resistance of the urethra. Chronic over-exertion causes the muscular walls of the urinary bladder to remodel and become stiffer. This combination of increased urethral resistance to urine flow and stiffness and hypertrophy of urinary bladder walls leads to a variety of lower urinary tract symptoms (LUTS) that may severely reduce the patient's quality of life. These symptoms include weak or intermittent urine flow while urinating, straining when urinating, hesitation before urine flow starts, feeling that the bladder has not emptied completely even after urination, dribbling at the end of urination or leakage afterward, increased frequency of urination particularly at night, urgent need to urinate, etc.

Although BPH is rarely life threatening, it can lead to numerous clinical conditions including urinary retention, renal insufficiency, recurrent urinary tract infection, incontinence, hematuria, and bladder stones.

Minimally invasive procedures for treating BPH symptoms include Transurethral Microwave Thermotherapy (TUMT), Transurethral Needle Ablation (TUNA), Interstitial Laser Coagulation (ILC), and Prostatic Stents.

In TUMT, microwave energy is used to generate heat that destroys hyperplastic prostate tissue. This procedure is performed under local anesthesia. In this procedure, a microwave antenna is inserted in the urethra. A rectal thermosensing unit is inserted into the rectum to measure rectal temperature. Rectal temperature measurements are used to prevent overheating of the anatomical region. The microwave antenna is then used to deliver microwaves to lateral lobes of the prostate gland. The microwaves are absorbed as they pass through prostate tissue. This generates heat which in turn destroys the prostate tissue. The destruction of prostate tissue reduces the degree of squeezing of the urethra by the prostate gland thus reducing the severity of BPH symptoms.

Another example of a minimally invasive procedure for treating BPH symptoms is TUNA. In this procedure, heat induced coagulation necrosis of prostate tissue regions causes the prostate gland to shrink. It is performed using local anesthetic and intravenous or oral sedation. In this procedure, a delivery catheter is inserted into the urethra. The delivery catheter comprises two radiofrequency needles that emerge at an angle of 90 degrees from the delivery catheter. The two radiofrequency needles are aligned are at an angle of 40 degrees to each other so that they penetrate the lateral lobes of the prostate. A radiofrequency current is delivered through the radiofrequency needles to heat the tissue of the lateral lobes to 70-100 degree Celsius at a radiofrequency power of approximately 456 KHz for approximately 4 minutes per lesion. This creates coagulation defects in the lateral lobes. The coagulation defects cause shrinkage of prostatic tissue which in turn reduces the degree of squeezing of the urethra by the prostate gland thus reducing the severity of BPH symptoms. In some cases, the , heat induced coagulation necrosis of prostate tissue regions is accomplished with steam.

Another example of a minimally invasive procedure for treating BPH symptoms is ILC. In this procedure, laser induced necrosis of prostate tissue regions causes the prostate gland to shrink. It is performed using regional anesthesia, spinal or epidural anesthesia or local anesthesia (periprostatic block). In this procedure, a cystoscope sheath is inserted into the urethra and the region of the urethra surrounded by the prostate gland is inspected. A laser fiber is inserted into the urethra. The laser fiber has a sharp distal tip to facilitate the penetration of the laser scope into prostatic tissue. The distal tip of the laser fiber has a distal-diffusing region that distributes laser energy 360° along the terminal 3 mm of the laser fiber. The distal tip is inserted into the middle lobe of the prostate gland and laser energy is delivered through the distal tip for a desired time. This heats the middle lobe and causes laser induced necrosis of the tissue around the distal tip. Thereafter, the distal tip is withdrawn from the middle lobe. The same procedure of inserting the distal tip into a lobe and delivering laser energy is repeated with the lateral lobes. This causes tissue necrosis in several regions of the prostate gland which in turn causes the prostate gland to shrink. Shrinkage of the prostate gland reduces the degree of squeezing of the urethra by the prostate thus reducing the severity of BPH symptoms.

In all these procedures, and in other procedures such as the placement of an implant in urethral or prostatic tissue, there is a need for providing information regarding the position of the treating instrument relative to important anatomical landmarks. The present disclosure addresses these needs.

### SUMMARY OF THE INVENTION

Certain aspects of the present invention are directed towards augmenting imaging data used in a medical procedure, such as diagnosis or treatment of urogenital diseases or conditions in a human patient Imaging data that is typically viewed by a treating physician during a procedure is augmented with information such as: identification of anatomical landmarks; identification of device features; relative distance between anatomical landmarks and device features; speed of device movement; stage of procedure; estimated percent or fraction of completion of the procedure; degree of tissue compression; and/or estimated efficacy of the procedure (such as the degree of opening of a lumen occlusion).

The system and method use a neural network deep learning model to process and augment cystoscopic images in real time. The system and method can be arranged in a set of modules, such as: a detection module; a segmentation module; a location measuring module; a speed measuring module; an action detection module; and/or a display module.

Other features and advantages of the present system and method will become apparent from the following description, taken in conjunction with the accompanying drawings, which illustrate, by way of example, certain principles of the system and method.

### BRIEF DESCRIPTION OF THE DRAWINGS

FIG. 1A is a cross-sectional view through the lower abdomen of a human male depicting a region of the urogenital system.

FIG. 1B is an enlarged cross-sectional view from FIG. 1A.

FIG. 2A shows a coronal section through the lower abdomen of a human male depicting a region of the urogenital system, including a section through the prostate gland, and a device and method for conducting a medical procedure in the region according to certain aspects of the invention.

FIG. 2B shows a coronal section through the lower abdomen of a human male depicting a region of the urogenital system, including a section through the prostate gland, and a device and method for conducting a medical procedure in the region according to certain aspects of the invention.

FIG. 3A is a side view depicting a treatment device according to the prior art.

FIG. 3B is an enlarged perspective view of a distal portion of a treatment device according to the prior art.

FIG. 4 is a representation of an image from an endoscope, where the image has been taken during a procedure and information has been processed and used to augment the image according to certain aspects of the invention.

FIG. 5 is a flowchart depicting a method according to certain aspects of the invention.

### DETAILED DESCRIPTION OF THE INVENTION

Turning now to the figures, which are provided by way of example and not limitation, the present disclosure is directed to a system and method for augmenting imaging data used in a medical procedure, such as diagnosis or treatment of urogenital diseases or conditions in a human patient. Advantageously, the system and method of the present invention, in some aspects, uses existing imaging modalities to provide positioning and targeting information not already provided by these modalities. In some instances, the system and method collect data from the imaging modalities, process that data, and display the processed results back to the user to improve the accuracy, efficacy, and/or safety of a therapeutic or diagnostic treatment.

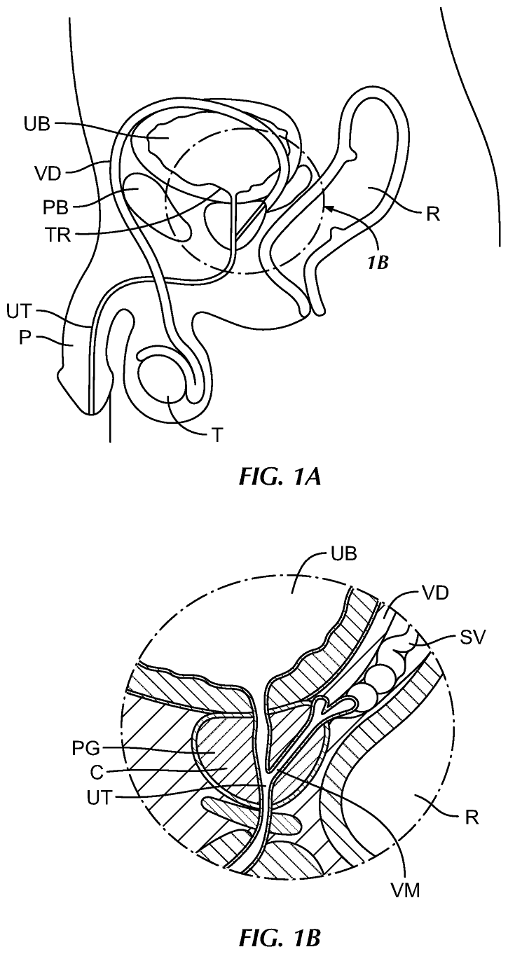

With reference to FIGS. 1A and 1B, various features of urological anatomy and genital anatomy of a human male subject are presented. The prostate gland PG is a walnut-sized muscular gland located adjacent the urinary bladder UB. The urethra UT runs through the prostate gland PG and connects the urinary bladder UB to the exit point for urine at the tip of the penis P. The prostate gland PG secretes fluid that protects and nourishes sperm. The prostate also contracts during ejaculation of sperm from the testes T to expel semen and to provide a valve to keep urine out of the semen. A firm capsule C surrounds the prostate gland PG.

The vas deferentia VD define ducts through which semen is carried and the seminal vesicles SV secrete seminal fluid. The rectum R is the end segment of the large intestine and through which waste is expelled. The urinary bladder UB is near the pubic bone PB. The urethra UT carries both urine and semen out of the body. Thus, the urethra is connected to the urinary bladder UB and provides a passageway to the vas deferentia VD and seminal vesicles SV.

Further, the trigone TR is a smooth triangular end of the bladder. It is sensitive to expansion and signals the brain when the urinary bladder UB is full. The verumontanum VM is a crest in the wall of the urethra UT where the seminal ducts enter. The prostatic urethra is the section of the urethra UT that extends through the prostate.

Several of the anatomical structures discussed above, including the prostate gland PG, can be treated using minimally invasive procedures. As illustrated in FIGS. 1A and 1B, there are multiple relevant anatomical landmarks that can be useful in determining the relative position of a minimally invasive device. For example, the verumontanum VM and the bladder neck (or junction of the urethra UT and the urinary bladder UB) can define the boundaries of a treatment region for treating BPH in the prostate gland PG.

Similarly, FIGS. 1A and 1B illustrate several anatomical areas that can complicate a procedure. For example, one complication of treatment of the prostate gland PG can be an inadvertent puncture of the rectum R. Another complication of treatment of the prostate gland PG can be inadvertent contact with the pubic bone PB.

Aspects of the system and method of the present invention as disclosed herein provide positional information to assist a user in locating the desired treatment areas and avoiding inadvertent contact with other parts of the patient's anatomy.

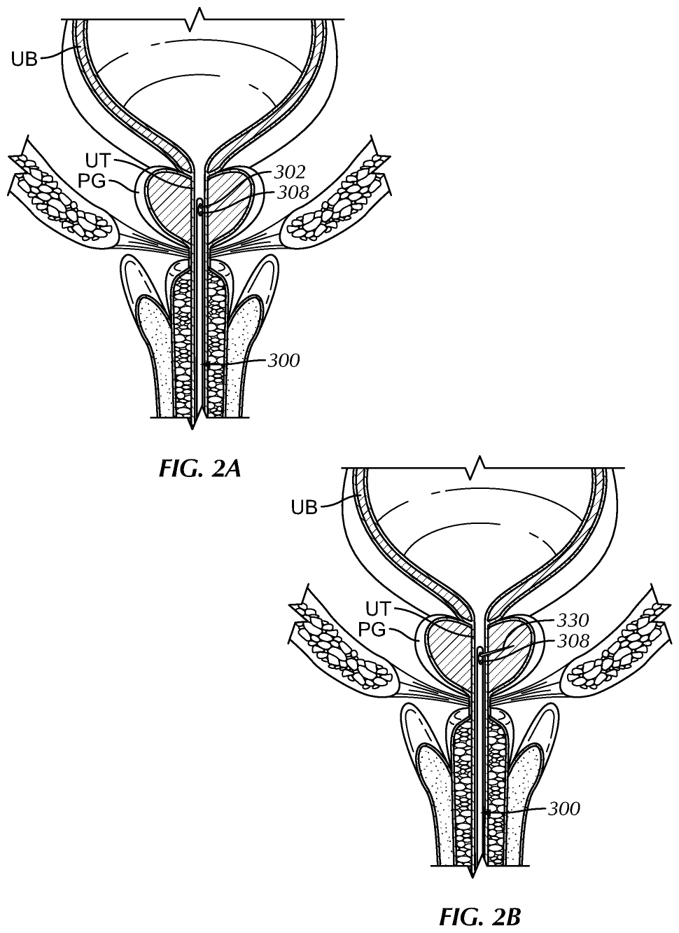

FIGS. 2A and 2B show a coronal section through the prostate gland PG showing some of the various steps of a method of treating prostate gland disorders. In FIG. 2A, an introducer 300 has been introduced into the urethra UT through the urethral opening at the tip of the penis. A cystoscope is inserted in the introducer 300 through the cystoscope lumen 308 such that the lens of the cystoscope is located near a distal opening of the cystoscope lumen. The cystoscope is used to navigate the introducer 300 through the urethra such that the distal region of the introducer 300 is located in a target region in the prostatic urethra. Thereafter in FIG. 2B, an injecting needle 330 is advanced through the working device lumen 302 such that the distal tip of the injecting needle 330 penetrates into a region of the urethral wall or the prostate gland. The injecting needle 330 is then used to inject one or more diagnostic or therapeutic agents into the urethral wall or the prostate gland. This step may be repeated one or more times to inject one or more diagnostic or therapeutic agents in one or more regions of the urethral wall and/or the prostate gland. In another embodiment, injecting needle 330 is used to deliver energy such as, but not limited to, radiofrequency energy, heat energy, laser energy, or microwave energy.

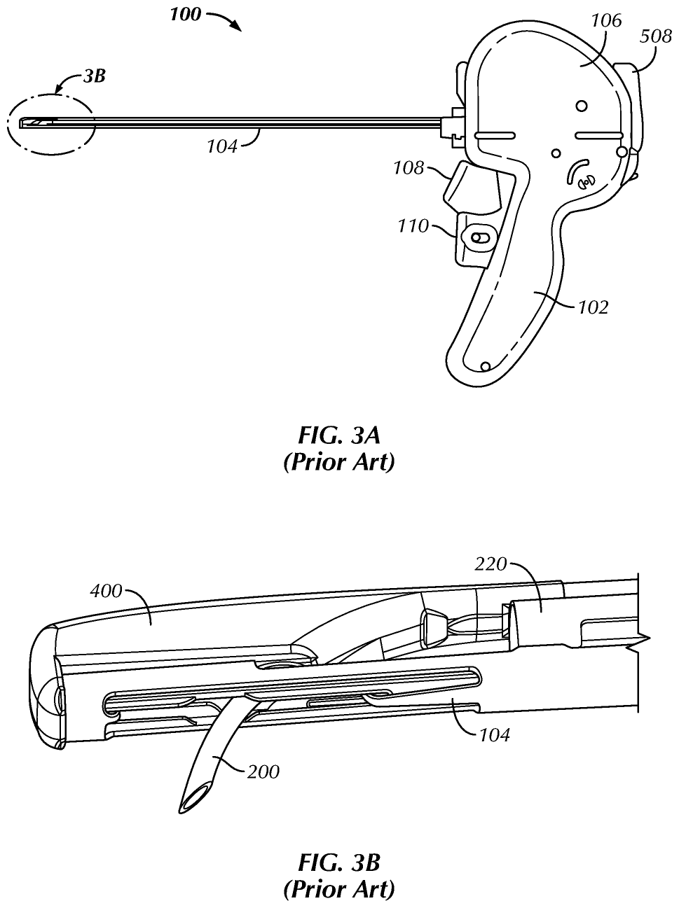

FIGS. 3A and 3B show a delivery device 100 known in the prior art. This device and its methods of use are disclosed in U.S. Pat. Nos. 7,645,286; 7,758,594; 7,766,923; 7,905,889; 7,951,158; 8,007,503; 8,157,815; 8,216,254; 8,333,776; 8,343,187; 8,394,110; 8,425,535; 8,663,243; 8,715,239; 8,715,298; 8,900,252; 8,936,609; 8,939,996; 9,320,511; 9,549,739; 10,105,132; and 10,299,780. This device is configured to include structure that is capable of both gaining access to an interventional site as well as assembling and implanting one or more anchor assemblies or implants within a patient's body. The delivery device 100 can be configured to assemble and implant a single anchor assembly or implant a single bodied anchor or multiple anchors or anchor assemblies. The device can be compatible for use with a 19F sheath. The device additionally includes structure configured to receive a conventional remote viewing device (e.g., an endoscope) so that the steps being performed at the interventional site can be observed.

The anchor delivery device 100 includes a handle assembly 102 connected to elongate member 104. Elongate member 104 can house components employed to construct an anchor assembly and is sized to fit into a 19F cystosopic sheath for patient tolerance during a procedure in which the patient is awake rather than under general anesthesia.

The elongate member 104 of the delivery device is placed within a urethra UT leading to a urinary bladder UB of a patient. The delivery device can be placed within an introducer sheath previously positioned in the urethra or alternatively, the delivery device can be inserted directly within the urethra. The elongate member 104 is advanced within the patient until a leading end thereof reaches a prostate gland PG. In a specific approach, the side(s) (or lobe(s)) of the prostate to be treated is chosen while the device extends through the bladder and the device is turned accordingly.

Upon depression of the needle actuator 108, the needle assembly 200 is advanced from within the elongate member 104. The needle assembly can be configured so that it curves back toward the handle as it is ejected. In use in a prostate intervention, the needle assembly is advanced through the prostate gland (PG) and an anchor assembly is deployed. Typically, more than one anchor assembly is deployed during an intervention.

In order to view this operation, the delivery device 100 can be provided with an endoscope 220 that can be placed within a lumen in the delivery device. The endoscope 220 allows the user to view the region of the distal end 400 of the delivery device as well as tissue near the delivery device.

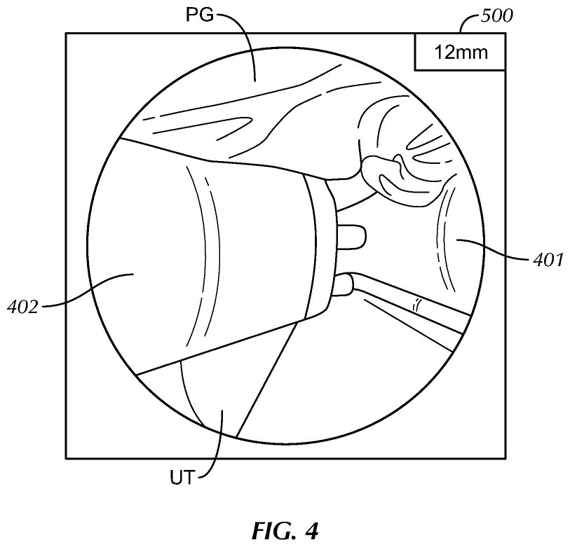

FIG. 4 is an image from an endoscope, where the image has been taken during a procedure and information has been processed and used to augment the image. FIG. 4 shows the lower portion 401 of the distal end 400 of the delivery device and the upper portion 402 of the distal end 400 of the delivery device as viewed through the endoscope mounted inside the delivery device. In this image, the delivery device has been rotated 90 degrees from its upright position such that the distal end of the device is positioned against a lateral lobe of the prostate gland. The needle assembly will advance from the lower portion 401 and penetrate the lateral lobe of the prostate gland on the viewer's right in the image shown in FIG. 4. The prostate gland PG identified in FIG. 4 is part of the lateral lobe that is being treated. A portion of the urethra UT is visible at the bottom of the image.

FIG. 4 also shows a position indicator 500, which is displayed on the image that is visible to a user on the imaging equipment. The position indicator 500 in this embodiment shows the distance from the bladder neck to the point on the delivery device from which the needle assembly 200 exits the delivery device. This distance has been calculated by the system and method of this example to be 12 mm.

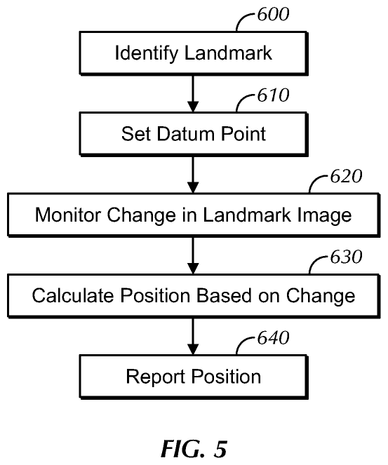

FIG. 5 illustrates a flowchart of one embodiment of the system and method of the present invention. In step 600, a relevant anatomical landmark is identified. The identification of the landmark can be automatic, manual, or a combination in which the system prompts the user to confirm the identity of a suggested landmark, where the suggestion is made by the system. The system can be trained on a database of videos and/or images to recognize various known anatomical landmarks relevant to a particular procedure. For example, the system can be trained on a database of relevant videos and/or images to identify the verumontanum and the bladder neck and to prompt the user to confirm when those landmarks appear at a specific place in the image.

In step 610, a datum point is set based on the identified landmark. A datum point is the reference from which other positions are determined. For example, the system can set the datum point at the bladder neck for procedures where the location of the bladder neck defines the relative position for an intervention.

In step 620, the image of the landmark, or the area near the landmark, at which the datum point is set is monitored for changes. The changes in the image are created as the endoscope and delivery device move relative to the landmark. The brightness, contrast, or focal quality of the landmark may change as the endoscope and delivery device move relative to the landmark. The perceived size of the landmark in the image may change as the endoscope and delivery device move relative to the landmark. The system can be trained on a database of relevant videos and/or images showing how the perceived image of various landmarks will change based on the movement of the endoscope and delivery device relative to the landmark. The system can be trained on how various lighting and lens combinations will display the anatomical landmark based on the movement of the endoscope and delivery device relative to the landmark. In some aspects, the identity of the endoscope will be provided to the system. In other aspects, the system includes a calibration structure and method for calibrating how the lighting and lens combinations react to a known image and these measured values can be provided to the system.

In step 630, the system calculates a position on the delivery device relative to the datum point based on how the perceived image of the landmark changed. The system can be trained on how to convert image changes to distance values using a database of relevant videos and/or images, a calibration structure, or both. The calibration of the system can include using known dimensions on the treatment instrument. That is, the perceived size in the image of a feature of known dimension can be used to inform the calculation of distances based on image changes. Referring again to FIG. 4, the dimension of the point where the lower portion 401 of the distal end 400 of the delivery device meets the upper portion 402 of the distal end 400 of the delivery device is a finite, known dimension. The size of that dimension can be used in the calculation of the dimension of other image features. Thus, the system and method can be adapted to various therapeutic devices and methods by using fixed dimensions specific to the therapeutic device.

In step 640, the position is reported to the system, the user, or both. The position can be displayed on the screen of the imaging equipment as a number or through graphical representations such as progress along a bar, dial, or the like. In some aspects, the graphical representation has a target point along the bar, dial, or the like to indicate a destination position for the device.

In some aspects, steps 600 through 640 are repeated multiple times in a given procedure. In such embodiments, the datum point may remain the same, or the datum point may be recalculated each time through the steps. For example, after an anchor has been placed, the location of that anchor may be the next datum point.

Some aspects of the system and method of the present invention calculate the speed of the device movement via monitoring the rate of change of the perceived image of the relevant landmark as a function of time. In certain procedures, it is beneficial to the procedure and/or the patient for the device to move below a certain speed threshold. For example, higher speed device movements may cause damage (or more severe damage) to the lining of the urethra as compared to lower speed device movements. As another example, in procedures that use energy to modify tissue there may be a desired range of speeds for moving the instrument that delivers the energy to optimize the procedure. In some embodiments, there is a signal to the user to provide guidance to the user that the device is being moved outside the desired speed range. The signal can be a visual cue on the image display, an audible cue generated by the system, and/or a tactile cue on the delivery device.

In some aspects of the system and method, a surrogate image for the delivery device and/or other features of interest (such as suggested locations for implant(s)) is placed on the pre-procedure image. The display screen can include other indicia, such as estimates of procedure progress and estimates of the volume of free space in the urethra before, during, and after the procedure.

In some aspects of the system and method, the contrast in the image can indicate the degree of tissue compression. That is, when tissue is compressed, the blood is squeezed out of the local tissue area and this change in local blood content causes the tissue to appear whiter in the image than prior to compression. By measuring this contrast change, the degree of compression can be calculated or estimated.

The system and method can be arranged in modules, including, but not limited to: a detection module for detecting device features and/or anatomical features; a segmentation module for segmenting device features and/or anatomical features, a location measuring module for determining the relative location of device features; a speed measuring module for determining the relative speed of device features; an action detection module for identifying different stages in a treatment; and a display module for displaying measured information to the user. Any of the attributes and/or functions of a module may be combined with attributes and/or functions of any other module such that the single combined module exhibits the attributes and/or functions listed above as separate modules.

Detecting features involves identifying the general boundary areas of the feature within the image. Segmenting the anatomical features involves identifying the specific boundaries of the anatomical feature within the general boundaries identified in the detection step. In the context of medical imaging, segmentation is valuable for reinforcing a deep learning model that allows a system and method to correctly detect and segment anatomical features and/or treatment device features across of variety of presentation instances in a variety of images and image contexts.

In certain aspects of the system and method disclosed herein, anatomical features of the relevant treatment area are detected and segmented from device features concurrent with the treatment. In certain aspects, the anatomical features of interest include, but are not limited to, the bladder, the bladder neck, the urethral walls, the urethral lumen, and the verumontanum.

Parts of the treatment device are visible in imaging during the treatment, and certain features of the treatment device are detected and segmented concurrent with the treatment. By detecting and segmenting device features, the system and method disclosed herein can monitor the stages of the treatment and provide information to a user regarding such stages. In certain aspects, the device features include, but are not limited to, the distal end of the device, the suture, and the urethral endpiece (which is a part of an anchor assembly placed in tissue via a delivery device).

In certain aspects of the system and method disclosed herein, the speed of the device is calculated using optical flow fields. These calculations account for the environment in which the device is moving, which is distensible due to the elastic mechanical properties of the prostatic urethra.

In some aspects, the system and method for detection and segmentation uses template matching to detect device features via: creating a rectangular mask with the device estimated to be centered in the mask; creating a rectangular image using a centered endoscopic view; finding the transformation that minimizes the cross-correlation between the images; and applying the inverse transformation on a binary mask of the device. In some instances, color models are used to segment device features.

One aspect of the system and method is the ability to detect different stages of the treatment process. One example of a treatment stage is the point in a BPH treatment where an implant has been completely deployed and disconnected from the treatment device. This stage is identifiable by a difference in the appearance of a distal portion of the device in the image. That is, a specific change to a device feature indicates that the treatment stage has been reached. Thus, detecting device feature changes can be a function of an action detection module

As one example of a detection and segmentation module, a deep learning model is given an image to calculate complex features about the content of an image, and then outputs a vector of numbers such that each number represents whether or not a device feature (or anatomical feature) is present. The deep learning model can detect whether there is a specific device feature, such as a cutting assembly distal end portion, present in the image. Image filters and image noise reduction methods can improve the accuracy of the deep learning model. Another example of a specific device feature is the uncut suture, which can reflect the light from the endoscope in a specific manner (e.g., a “white line” appears along a portion of the length of the suture) when the suture is in the proper position for implant deployment.

When detecting and segmenting device features and anatomical features, it is possible to assist the model by making certain informed, reasonable assumptions about the images. For example, when identifying the verumontanum, it is reasonable to bias the model to look for distinguishing features of the verumontanum in the bottom-middle portion of the cystoscopy image because of the known typical angle of entry and patient position during the cystoscopy. Further, image gradients may be useful in identifying the verumontanum. Certain image attributes are helpful in detecting and segmenting the verumontanum, including, but not limited to,: the verumontanum segmentation center of mass; the verumontanum segmentation width, height, and size; the ratio of the segmentation border on the tissue border; and the ratio of segmentation border on the device border.

As another example, certain device features are known to be present on only one side of the treatment device. Thus, after detection and segmentation to determine device orientation, the model can be biased to detect and segment certain device features on one side or the other of the device.

In some aspects of the system and method, a voting queue is used to allow the neural network of the algorithm to vote on whether a device or anatomical feature is present in the image. If a certain voting threshold is reached, then the system will accept the results of the majority vote. For example, if in 6 out of the last 11 frames the network votes that the distal end of the device appears to be in the bladder, then the location of the distal end of the device is set to the bladder. As another example, a voting queue can be used to determine whether the device is adjacent the urethral wall. This process can reduce and/or eliminate false detections of certain important anatomical features and allow for a lower threshold of certainty when using the network analysis.

In some aspects of the system and method, a display module receives an image from the imaging equipment and annotates the image with information to display on a screen for the user. The display module can include an action extraction module, which receives a video file and annotates the file (e.g., writing a new file with the original information and including additional information) with additional information such as, but not limited to, the action name, the image frame number in which the action occurred, and the location of the action with respect to an anatomical feature. One example is that the action “attaching the urethral endpiece” occurred in frame 2283 at 16 mm from the bladder opening.

In some aspects of the system and method, it is useful to detect whether the BPH treatment device is sticking to the urethral wall. If the condition where the BPH treatment device is stuck to the urethral wall, the apparent speed of the device may be measured as artificially high due to the optical flow field changing due to tissue distortion rather than actual device movement. In this condition, the speed estimate must be corrected. Further, it is useful to inform the user that the BPH treatment device is close to the urethral wall so that the user can minimize tissue damage due to the device abrading tissue.

In some aspects, the system and method uses a simultaneous localization and mapping algorithm, which has three principal parts: feature detection, feature mapping, and translation calculation. One approach to the simultaneous localization and mapping algorithm includes preprocessing images to convert the images to formats that allow for easier feature extraction. For example, an image that includes views of the urethral wall can be converted into a map of the veins present in the urethral wall. In some cases, a filter similar in functionality to a frangi filter, which is known and used for identifying vasculature in angiographic imaging, can be used to map the veins present in the urethral wall. In some cases, it is useful to add a depth map to the simultaneous localization and mapping algorithm to increase the accuracy of the algorithm.

A particular aspect of the simultaneous localization and mapping algorithm is the assumption that the real-world coordinates of the detected features are inside an elastic cylinder and the image viewer is within the cylinder. This distinguishes the simultaneous localization and mapping algorithm of this system and method from other imaging algorithms where the real-world coordinates are projected onto a conventional three-dimensional space. The cylindrical assumption enables the simultaneous localization and mapping algorithm to filter out feature positions that are inconsistent with a cylindrical coordinate system.

Another aspect of the simultaneous localization and mapping algorithm is that it is possible to assume the distance between points of view in each image is comparatively small and that the viewing planes are more or less parallel to each other. This assumption allows for a low feature similarity threshold while still filtering out incorrect feature matches in a time period consistent with the needs of the procedure.

In some aspects, the system and method uses a speed measuring module for determining the relative speed of device features and a location measuring module for determining the relative location of device features. Having identified features using a detection and segmentation module, strong features (that is, features whose imaged attributes make them easy to distinguish from the image background) make it possible to create a uniform measure of distance (location) with the speed module and frame count.

In some aspects, the system and method uses a Direct Sparse Odometry (DSO) algorithm to identify many (e.g., a few thousand) interest points in the image and to estimate the distance of these points of interest from the point of view in the image. For example, using several consecutive image frames the DSO algorithm tracks the movement of the interest points and, based on their movement, changes the estimate of the location of the point of view.

In some aspects, the system and method uses a Structure From Motion (SFM) algorithm, which is a similar method to DSO of creating a scene from still images. The algorithm uses a set number of images, calculating the movement in the last two images in the set and then another image is added to the set and the process repeats.

In some aspects, a process of relocalization is used to reset the location of the model. During real-time image collection process, the SFM algorithm may lose track of location in certain frames due to reasons such as blurring in the images, too fast movement of the device, or blood or other debris obscuring the image. Once the quality of the image frames improves, it is necessary to be able to connect these image frames with the last set of “good” image frames. To accomplish this, a descriptor can be added to each frame where the descriptor is based on an image attribute such as the RGB color histogram. Using the descriptor, frames having similar attributes can be located and the similar frames matched for localization purposes. In some cases, it is useful to add a fast filter to the SFM algorithm to check which images are sufficient quality to be added to the model.

In some cases, a weighted sum of previous location predictions can be used to predict the location of each incoming frame and improve the module. And, an image similarity score can be used to improve localization.

In some cases, a location module can use the speed estimate to improve localization. That is, if the measured speed is high, multiple new locations can be added to the location calculation. If similarity scores between image frames are poor, the speed estimate can be used to calculate location.

In some aspects, the speed measuring module uses an optical flow field with patch-wise constraints. In other aspects, the speed measuring module uses transformation calculations (such as translation, rotation, and scale) by using strong features and feature matching. Both of these methods create vector fields, which allow for measurements of speed per frame based on vector magnitude.

In some aspects of the system and method, the process for extracting movement direction and speed from a dense optical flow field begins with the steps of: filtering out all small vectors; performing a transformation; and taking the maximum of the transformation to obtain the focus of expansion. Then, if the maximum is above or below a predefined threshold, the direction is either into or away from an anatomical feature (such as the bladder).Once the direction of movement is determined, the optical flow vectors that intersect the focus of expansion can be used to calculate the speed by taking the average of the magnitude of all these intersecting vectors.

An SFM algorithm can be used to convert a sequence of images into a sequence of location and speed measurements. A sequence of images is sent to the SFM algorithm where, each time an image is removed from the beginning of the sequence, a new one is added to the end. The results are processed to get a point of view location in each scene. The last point of view location is compared to the previous point of view location to calculate the transformation vector and therefore the speed and the direction of movement. Using pairs of known points that are mutual between two consecutive models, it is possible to calculate a linear transformation from one model to another model and preserve angles and distances. This transformation is used to align both models and only then calculate the translation vector between the new image and another previously calculated point. Thus, both the magnitude and direction of the movement in a sequence can be tracked.

In some cases, at very high actual device movement speeds, the speed calculation can fail so the system and method can indicate to the surgeon the movement is too fast for location tracking.

The system includes a microprocessor and addressable memory configured to perform the calculations disclosed herein and an input/output interface configured to gather image data from the imaging system and send calculated data for display on the imaging system. The system and method are configured to be compatible with various video imaging systems.

The relevant positional information may vary from procedure to procedure and it should be understood that other anatomical landmarks than the bladder neck and verumontanum are included in this disclosure. Similarly, the relevant device feature may vary from procedure to procedure and the disclosure is not limited to the examples of relevant device features specifically listed herein. In some aspects, relevant physical features related to the device include the exit point of the needle from the device and the entry point of the needle into tissue, which may be different from each other. In some aspects, the system and method provide a needle position image superimposed over a pre-procedure image of the prostate collected via the various known ways of collecting such an image, such as magnetic resonance imaging, computed tomography, and/or ultrasound.

Using information gathered about the patient's anatomy via magnetic resonance imaging, computed tomography, and/or ultrasound, the system and method may provide the user with warnings and/or predictions regarding the possibility of inadvertent contact with anatomical features. For example, the system and method may predict the possibility that the needle assembly will strike bone if deployed at a certain position or angle relative to the datum point using the information gathered about the patient's anatomy and the positional information gathered via the method illustrated in FIG. 5. In this way, the system and method can be used to optimize clinical efficacy, improve implant placement consistency, and reduce the incidence of undesirable outcomes.

As another example of the system and method providing the user with warnings and/or predictions, the system and method may predict that a user has reached a particular stage in the procedure even though the typical visual cues available to a user may not be present or obvious to the user. In one instance of this example, the white line (that is, the reflection of light on the suture as described previously herein) that indicates that the procedure has reached the stage where the suture can be cut may not be visible to the user because something is blocking the view of the suture, such as bulging tissue or a bubble in the field of view. The system and method can analyze the motion of the device with respect to the urethra based on the modules disclosed herein and make inferences about the stage of the procedure. Predicting the presence of the white line can be done by measuring how much the user compresses tissue during an earlier stage of the procedure and noting that the needle was advanced by observing changes in the image of the distal portion of the device. With the information about the location where the needle was advanced, the system can calculate how much the device has to move in order for the suture to be in a position to create the light reflection (i.e., the white line). Thus, the system and method can predict that the white line will be visible and can confirm this by detecting the white line when it becomes visible. If the white line does not become visible, the user will still have the information that the device is in the proper location for the stage of the procedure where the suture is cut. Other procedure information can be used to predict other parts of the procedure. And more broadly, other stages of other procedures may be predicted using a similar system and method as disclosed herein.

One example of the invention is a method for providing relative positional information to a user during a procedure employing a therapeutic or diagnostic device. The method includes the steps of identifying an anatomical landmark near a target site for the device in an image of the procedure that includes the device in the image; setting a datum point based on the anatomical landmark; monitoring a change in the anatomical landmark as shown in the image as the device is moved; calculating the relative positional information based on the change using a microprocessor; and reporting the relative positional information.

The method can include the step of measuring a known distance in the images to calibrate the microprocessor.

The method can include the step of training the microprocessor using a database of relevant data.

The method can include the steps of collecting other anatomical information and combining the positional information with the other anatomical information and displaying the combination.

The method can include the step of predicting possible inadvertent anatomical contacts using the combination of positional information with the other anatomical information.

The method can include the steps calculating speed information related to device movement and providing the speed information to the user via visual, audible, or tactile signals.

Another example of the invention is a system for providing relative positional information to a user during a procedure employing a therapeutic or diagnostic device. The system can include: a delivery device carrying an implant delivered through a needle; an endoscope mated to the delivery device; an imaging system operatively connected to the endoscope; and a microprocessor and addressable memory operatively connected to an input/output interface. The input/output interface is configured to be connectable with the imaging system and the microprocessor and addressable memory calculate relative positional information using a plurality of images displayed on the imaging system where at least one of the plurality of images includes an anatomical landmark and the device.

While particular elements, embodiments and applications of the present invention have been shown and described, it will be understood that the invention is not limited thereto since modifications can be made by those skilled in the art without departing from the scope of the present disclosure, particularly in light of the foregoing teachings.

1. A system for providing treatment information to a user during a minimally invasive procedure, comprising:
a therapeutic or diagnostic device;
an endoscope mated to the device;
an imaging system operatively connected to the endoscope; and
a microprocessor and addressable memory operatively connected to an input/output interface, wherein the input/output interface is configured to be connectable with the imaging system;
wherein the microprocessor and addressable memory calculate treatment information using a trained algorithm and display the treatment information on the imaging system.

2. The system of claim 1, wherein the therapeutic or diagnostic device comprises a needle;

3. The system of claim 1 wherein the treatment information comprises a distance between an anatomical landmark and a device feature.

4. The system of claim 3 wherein the anatomical landmark is a bladder opening and the device feature is a needle exit point.

5. The system of claim 1 wherein the treatment information further comprises a speed measurement of the device.

6. The system of claim 1 wherein the treatment information further comprises a stage of the procedure.

7. The system of claim 1 wherein the treatment information further comprises an estimated percent or fraction of completion of the procedure.

8. The system of claim 1 wherein the treatment information further comprises a degree of tissue compression.

9. The system of claim 1 wherein the treatment information further comprises an estimated efficacy of the procedure.

10. The system of claim 1 wherein the treatment information further comprises a warning of inadvertent anatomical contact by the device.

11. The system of claim 1 wherein the treatment information further comprises guidance about the device speed.

12. The system of claim 1 wherein the algorithm includes one or more of a detection module, a segmentation module, a location measuring module, a speed measuring module, an action detection module, and/or a display module.

13. The system of claim 1 wherein the algorithm is trained using a Direct Sparse Odometry (DSO) algorithm.

14. The system of claim 1 wherein the algorithm is trained using a Structure From Motion (SFM) algorithm.
